SEQUENCE LISTING (11 sequences)

SEQ ID NO: 1 - DNA, 1280 bp, Sorghum bicolor
ttacaatagacacatctaagttgggttaatcttataattatgtcctgttcgtttggctgataagtcataacaaaaaatattcttgattaattcattgtgagagaaaaatactactgaatagttggcagatttggctgataagcacaagcaaacaggcttaagtttttttatatgagattaatcctttatattgcactactatttgttacagatattgaaattcattggttagatcaaaaccagtaattaagttcaccaaaaatagctagtttaactattattgattcgtgtacggcggagtaaggccttgtttagatccgaatttttttttttagattttgacactgtagcactttcatttttatttgacaaacattatccaatcatggagtaactaggtttaaaagattcatctcacaatttacaggcaaattgtgcaattagtttttgttttcatctatatttaatgcttcatgcatgtgccggaaaattcgatgtgacaaggaatgttgaaaagtttttggtttttgggtcaactaaacaaggcctaaattttgctgtgcacaaatgaaccttggacctatggtgaaaagattcttggatgttccattacacaacatgcactaggtttactatggatcatggactagaagtagaagaaaacagatttgagtaatggatcttcatccaacggttagaatccaaccattttaagcactcaactattgtgtagacagactcaaattttaacatgaggagcgacaatgactctgttcattcttctaagtcgtacttttttctactagctagtagagtttttctttcacaataaatgaatgaacaatttttagttatagcttttcagataaacgaatacgctcggaacgattcaattcaaccacgaggatagttccactcgactctattcattatccaattccattgagaatcaaataactatcgaaaagaaattccatccaagcgagcaaataaactaagggaggaaaaagatttaaaaaaatgaaaaaagcctccgtcgcgataggtcggcaggaatcgagagccctacagtagagtcgtgtggaaatgcataattagccagcttccttcggaacggcggttcgcccacctaattatacgcaccacacgcctataaagccaacaaacccgcagaggggctcaagccagacagctcctgcttcctcctggtcaagtcttatccgcctctcgtcaccaaccgcgtgcgctccacgcctcctccaggtccaggaaagcgagag

SEQ ID NO: 2 - DNA, 1228 bp, Setaria italica
aggcttaatctcgctagttacagaaacccaccggcttaatctgtgaattgtaacgctggcttaatctcaaactacataaatgttctacttctcgtgcatgtttcatagtggtgttcggatgttcccccatcttgatactcggaagcgtcaaactagtgattgaatgtcatgcttaaaacatttatgtagtttgagcaccacttacgatgaggcgagatgcccagtatgcgcaacaatttctcatcgaattatcatgtgacagctgtgatgattcgtggatgttgctcggatttcagagtatgctgcacgcaacttgcaaagcaatggcgccaggtgcatgcgtatcggtaggagaacggatatggccttggtctggaatctgctgcttttgttatacttgatgcgacataggctcatgctctcgatattcccctacggtcccggggtttacatttcttctacagcaatgccagattggcagattcggagaaaatgttacctgagcagtttaagctgtgttcttgcaatgatcatgacaccgtgatgatacgagagccgtgccgatgctttgcttttccttttttaggaaatgccaaatgccgatgctgttgctagtgcgccgcgacctcagcgcctggccccactagccaatgtgggtcagtgggcctcatttgaccttcaaacacttgtttggcccgtgcatgagttggcctcaacttcttattatcctgcacattcctttcaaaatgtatatttttcttgcacatttcctttaaaaactagcactattatcttgcatatctgtatattttagcaatgatctggttcttttaaaaaatttcaactcatggagaatgagccaacacatgattgacgtgaacgatcaaagtgtttaacgtcaaccgtcaattgacgaggatacaggctgtattcactacatccagaaatataataaataatataagagaggaaaaagataaaaaaaagcctccgtctcgataggtcggcaggaatccagagccctagccgtgtgggggatgcataattagcaagcgtcccccggaacggcggttcgcccacctaattctacgcaccgcgcgcctataaaaccaaccaagccgggcggaggggtggaaacgggacagctcctggtcaagtcttatccgcctctcgcctccaaccgcgtgcgctccacgccggctctctccctcctcccggcaagagaaag

SEQ ID NO: 3 - DNA, 651 bp, Sorghum bicolor
gtgagcacatccccctttcccctctcttcgattcaatccgtcgtcgtctcgatttggggatctggaggggttcgatcgcttgtcgccgcgaatcggctttggtatagccaacggatctcggatcgtagtcttcagaaggtccctagttttgcgatgtgttagtttttgccctatgattcttagatagaggggatgattgttcacgacctctgggctgtggggcggttgttgaatcgattggatctgttcgggatggtgagcacagctgggttttcgggggggaatttttatgccttgcggtcactctgttgaatcatggggtttcgacccgattcgtaggctgctccttgttttggatgcaagtaggcgtgtttgtagcgttcgggctccgattcgttatcagggattagaagacctgggatgggatttgaggaaatctggatattagtctagcacgtttctagttctattgttatatgagagtgattttggaaccctagggtatgtttgaccggtgtacggtgtagatgattgtccctaactgctgactgctaccgtcctacctctgtatcatcgatctgtgctagtttctgtttatttctcaaatgtttcggcttgtgtagcatgtgattgatagtatgattttggcag

SEQ ID NO: 4 - DNA, 701 bp, Setaria italica
gtgagcaccaacgccctttccccctcccccttctccctgattcaatccgcctcgttcttagcccgtgggattaggggatctggagggtttcgatcgcgtcacgcctcgaattagctctgcgaacgaacggatctcggatcgtagtctccgggatgcgtgcgagtttccccctatgattcgtatgattggtatggttgtctgttctcggcgtctgggtcgcggttgatgaattgattggagtcctttgggagggtgagcggctggatttttggggaaatttttgtgccttgtggccgctctgggggatcatggggttttgatccgattcatcgtgctccctgttgtggatgagaagtaggcgtgtttctagtctcctgacttcgatttgtccacagcggttagaaggatttagggaattttcgtttagcccctttctggatctactatctaggctgttgtatgagtaaaattttggaaccctaaagtttgaccgagaaaattgtggaactgtagtttcagtggtagtatttaacggattgacagctatggacgattgtcttatattgtactctctacttgttattgtgctttgctgctaccgttgaatactgtttctggcgatctgtgctagtttctatttcctcaaaatattttgtgcttgtatagcctgcgattgatggtattgtttgctttatgtgcag

SEQ ID NO: 5 - DNA, 2001 bp, Sorghum bicolor
ttacaatagacacatctaagttgggttaatcttataattatgtcctgttcgtttggctgataagtcataacaaaaaatattcttgattaattcattgtgagagaaaaatactactgaatagttggcagatttggctgataagcacaagcaaacaggcttaagtttttttatatgagattaatcctttatattgcactactatttgttacagatattgaaattcattggttagatcaaaaccagtaattaagttcaccaaaaatagctagtttaactattattgattcgtgtacggcggagtaaggccttgtttagatccgaatttttttttttagattttgacactgtagcactttcatttttatttgacaaacattatccaatcatggagtaactaggtttaaaagattcatctcacaatttacaggcaaattgtgcaattagtttttgttttcatctatatttaatgcttcatgcatgtgccggaaaattcgatgtgacaaggaatgttgaaaagtttttggtttttgggtcaactaaacaaggcctaaattttgctgtgcacaaatgaaccttggacctatggtgaaaagattcttggatgttccattacacaacatgcactaggtttactatggatcatggactagaagtagaagaaaacagatttgagtaatggatcttcatccaacggttagaatccaaccattttaagcactcaactattgtgtagacagactcaaattttaacatgaggagcgacaatgactctgttcattcttctaagtcgtacttttttctactagctagtagagtttttctttcacaataaatgaatgaacaatttttagttatagcttttcagataaacgaatacgctcggaacgattcaattcaaccacgaggatagttccactcgactctattcattatccaattccattgagaatcaaataactatcgaaaagaaattccatccaagcgagcaaataaactaagggaggaaaaagatttaaaaaaatgaaaaaagcctccgtcgcgataggtcggcaggaatcgagagccctacagtagagtcgtgtggaaatgcataattagccagcttccttcggaacggcggttcgcccacctaattatacgcaccacacgcctataaagccaacaaacccgcagaggggctcaagccagacagctcctgcttcctcctggtcaagtcttatccgcctctcgtcaccaaccgcgtgcgctccacgcctcctccaggtccaggaaagcgagaggtgagcacatccccctttcccctctcttcgattcaatccgtcgtcgtctcgatttggggatctggaggggttcgatcgcttgtcgccgcgaatcggctttggtatagccaacggatctcggatcgtagtcttcagaaggtccctagttttgcgatgtgttagtttttgccctatgattcttagatagaggggatgattgttcacgacctctgggctgtggggcggttgttgaatcgattggatctgttcgggatggtgagcacagctgggttttcgggggggaatttttatgccttgcggtcactctgttgaatcatggggtttcgacccgattcgtaggctgctccttgttttggatgcaagtaggcgtgtttgtagcgttcgggctccgattcgttatcagggattagaagacctgggatgggatttgaggaaatctggatattagtctagcacgtttctagttctattgttatatgagagtgattttggaaccctagggtatgtttgaccggtgtacggtgtagatgattgtccctaactgctgactgctaccgtcctacctctgtatcatcgatctgtgctagtttctgtttatttctcaaatgtttcggcttgtgtagcatgtgattgatagtatgattttggcagcttctgcataagatcaacgaatcaaagcgtgatcagctcggtgccgatcaaagatctacaaccaagttcc

SEQ ID NO: 6 - DNA, 2000 bp, Setaria italica
aggcttaatctcgctagttacagaaacccaccggcttaatctgtgaattgtaacgctggcttaatctcaaactacataaatgttctacttctcgtgcatgtttcatagtggtgttcggatgttcccccatcttgatactcggaagcgtcaaactagtgattgaatgtcatgcttaaaacatttatgtagtttgagcaccacttacgatgaggcgagatgcccagtatgcgcaacaatttctcatcgaattatcatgtgacagctgtgatgattcgtggatgttgctcggatttcagagtatgctgcacgcaacttgcaaagcaatggcgccaggtgcatgcgtatcggtaggagaacggatatggccttggtctggaatctgctgcttttgttatacttgatgcgacataggctcatgctctcgatattcccctacggtcccggggtttacatttcttctacagcaatgccagattggcagattcggagaaaatgttacctgagcagtttaagctgtgttcttgcaatgatcatgacaccgtgatgatacgagagccgtgccgatgctttgcttttccttttttaggaaatgccaaatgccgatgctgttgctagtgcgccgcgacctcagcgcctggccccactagccaatgtgggtcagtgggcctcatttgaccttcaaacacttgtttggcccgtgcatgagttggcctcaacttcttattatcctgcacattcctttcaaaatgtatatttttcttgcacatttcctttaaaaactagcactattatcttgcatatctgtatattttagcaatgatctggttcttttaaaaaatttcaactcatggagaatgagccaacacatgattgacgtgaacgatcaaagtgtttaacgtcaaccgtcaattgacgaggatacaggctgtattcactacatccagaaatataataaataatataagagaggaaaaagataaaaaaaagcctccgtctcgataggtcggcaggaatccagagccctagccgtgtgggggatgcataattagcaagcgtcccccggaacggcggttcgcccacctaattctacgcaccgcgcgcctataaaaccaaccaagccgggcggaggggtggaaacgggacagctcctggtcaagtcttatccgcctctcgcctccaaccgcgtgcgctccacgccggctctctccctcctcccggcaagagaaaggtgagcaccaacgccctttccccctcccccttctccctgattcaatccgcctcgttcttagcccgtgggattaggggatctggagggtttcgatcgcgtcacgcctcgaattagctctgcgaacgaacggatctcggatcgtagtctccgggatgcgtgcgagtttccccctatgattcgtatgattggtatggttgtctgttctcggcgtctgggtcgcggttgatgaattgattggagtcctttgggagggtgagcggctggatttttggggaaatttttgtgccttgtggccgctctgggggatcatggggttttgatccgattcatcgtgctccctgttgtggatgagaagtaggcgtgtttctagtctcctgacttcgatttgtccacagcggttagaaggatttagggaattttcgtttagcccctttctggatctactatctaggctgttgtatgagtaaaattttggaaccctaaagtttgaccgagaaaattgtggaactgtagtttcagtggtagtatttaacggattgacagctatggacgattgtcttatattgtactctctacttgttattgtgctttgctgctaccgttgaatactgtttctggcgatctgtgctagtttctatttcctcaaaatattttgtgcttgtatagcctgcgattgatggtattgtttgctttatgtgcaggttctgcataaagaccaacagatcaaagcctgatcggctcggtgccaatccaagatctacaaccaagttcc

SEQ ID NO: 7 - DNA, 70 bp, Sorghum bicolor
cttctgcataagatcaacgaatcaaagcgtgatcagctcggtgccgatcaaagatctacaaccaagttcc

SEQ ID NO: 8 - DNA, 71 bp, Setaria italica
gttctgcataaagaccaacagatcaaagcctgatcggctcggtgccaatccaagatctacaaccaagttcc

SEQ ID NO: 9 - DNA, 971 bp, Zea mays
agtcactcttcctttgatcattgtgccattgtaaaaaaaattatcatctctcagtagagagagtaattagttcattagctagtttaactcagctaataatttgttagatgactaattattggctgtaggattctaaacagagcctaaatagctggaatagctctagccctcaatccaaactaatgatatctatacttatgcaactctaaatttttattctaaaagtaatatttcatttttgtcaacgagattctctactctattccacaatcttttgaagctatatttaccttaaatctgtactctataccaataatcatatattctattatttatttttatctctctcctaaggagcatccctctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtttttcatccgccttcgcctccaaccgcgtgcgctccacgcctcctccaggaaagcgagag

SEQ ID NO: 10 - DNA, 893 bp, Zea mays
gtgagcgcagtcccctttcccctccttccaattcaattcgtcttctcgttcgcagccctaggatttgggggtctggaggggtttgatcgtttctcgccgtgaatctgctttggtgtaaaccaacggatctcggatcgtagtcttcagaagatcccggattttgcggtttggcccctcctggattcaattcgtcgtatcgttcgcagccctaggatttggggatctggaggggtttgatcgtttctcgccgcgaatctgctctggtgtaaaccaacggatctcgggtcgtagtcttcagaaggtcccggattttgcggtttggcccctcctggattcaattcgtcgtatcgttcgcagccctaggatttggggatctggaggggtttgatcctttctcgccgcgaatctgctctggtataaccaacggatctcgggtcgtagtcttcagaaggtcccggattttgcggtttggtggttcttgctctatgaatcagagggatggttcttcccggatttatgccttgcggccactctgtcgaatcatggggtttcgacccgattcgtaggcgtgctccctgttttggatgggaagtaggcgtgtttgtagtattcgtgcttcgattcgtcaacggagattagaagacctgggatgggatttgaggaaatctaggtatctgtctagcacgtttctagatctattcttcagctgttatatgagagtaattttggaaccctggtggggtatgtttgaccgagtattctgtagattattgtccgtgacttgctggctgttaccgtccttatctctgcatcatctatctgtgctagtttctgcgtgcttctcaaatatttccggcctgtgtagcatgtgactgataatatgattttggcag

SEQ ID NO: 11 - DNA, 69 bp, Zea mays
tctgcataagaacaacaaatcaaaagcttgatcagctcggtgcctacaaaacctcaacaaccaagtttc

(19) United States
(12) Patent Application Publication
(10) Pub. No.: US 2018/0305707 A1
(43) Pub. Date: Oct. 25, 2018

(54) GOS2 REGULATORY ELEMENTS AND USE THEREOF

(71) Applicant: PIONEER HI-BRED INTERNATIONAL, INC., Johnston, IA (US)

(72) Inventor: Shane Abbitt, Ankeny, IA (US)

(73) Assignee: PIONEER HI-BRED INTERNATIONAL, INC., Johnston, IA (US)

(21) Appl. No.: 15/738,033

(22) PCT Filed: Jun. 27, 2016
(86) PCT No.: PCT/US2016/039486

§ 371 Date: Dec. 19, 2017

Related U.S. Application Data
(60) Provisional application No. 62/185,209, filed on Jun. 26, 2015.

(51) Int. Cl. C12N 15/82; C07H 21/04

(52) U.S. Cl. CPC C12N 15/8216; C12N 15/8202; C07H 21/04

(57) ABSTRACT

The disclosure relates to gene expression regulatory sequences from Sorghum and Setaria, specifically to the regulatory regions of GOS2 gene and fragments thereof and their use in modulating the expression of one or more heterologous nucleic acid fragments in plants. The disclosure further discloses compositions, polynucleotide constructs, transformed host cells, plants and seeds containing the recombinant construct with the promoter, and methods for preparing and using the same.

### CROSS-REFERENCE TO PRIORITY APPLICATION

This application claims priority to U.S. Ser. No. 62/185,209 filed Jun. 26, 2015, the contents of which are hereby incorporated by reference in its entirety.

### FIELD

This disclosure relates to a plant regulatory elements and fragments thereof and their use in altering expression of at least one heterologous nucleotide sequence in plants.

### REFERENCE TO SEQUENCE LISTING SUBMITTED ELECTRONICALLY

The official copy of the sequence listing is submitted electronically via EFS-Web as an ASCII formatted sequence listing with a file named “6788USPSP_SequenceListing” created on Jun. 24, 2015 and having a size of 14 kilobytes and is filed concurrently with the specification. The sequence listing contained in this ASCII formatted document is part of the specification and is herein incorporated by reference in its entirety.

### BACKGROUND

Recent advances in plant genetic engineering have opened new doors to engineer plants to have improved characteristics or traits, such as plant disease resistance, insect resistance, herbicidal resistance, yield improvement, improvement of the nutritional quality of the edible portions of the plant, and enhanced stability or shelf-life of the ultimate consumer product obtained from the plants. Appropriate regulatory signals present in proper configurations help obtain the desired expression of a gene of interest. These regulatory signals generally include a promoter region, a 5′ non-translated leader sequence, an intron, and a 3′ transcription termination/polyadenylation sequence.

Plant derived constitutive promoters that have varying strength in expression are desirable to modulate the expression of a gene of interest.

### SUMMARY

A recombinant DNA construct includes a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence.

In an embodiment, a recombinant DNA construct comprises a polynucleotide sequence that has at least 95% identity, based on the Clustal V method of alignment with pairwise alignment default parameters (KTUPLE=2, GAP PENALTY=5, WINDOW=4 and

DIAGONALS SAVED=4), when compared to any of the sequences set forth in SEQ ID NOS: 1-8.

In an embodiment, a vector includes a the recombinant DNA construct having a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence. In an embodiment, a cell having a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence. In an embodiment, the cell is a plant cell.

In an embodiment, a plant includes a recombinant DNA construct having a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence. In an embodiment, the recombinant DNA construct is stably incorporated into its genome. In an embodiment, the plant is a monocot plant. In an embodiment, the plant is maize.

In an embodiment, a plant seed includes a recombinant DNA construct having a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence.

In an embodiment, a heterologous nucleic acid sequence that is operably linked to a fragment present in one of SEQ ID NOS: 1-8, comprises a genetic sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

In an embodiment, a recombinant DNA construct includes a polynucleotide sequence having any of the sequences set forth in SEQ ID NOS: 1-8, or a fragment that comprises at least about 50-100 contiguous nucleotides of SEQ ID NO: 1-6, operably linked to at least one heterologous nucleic acid sequence, wherein the heterologous sequence comprises a sequence that is substantially similar to an endogenous regulatory sequence of a maize gene.

In an embodiment, a method of expressing a coding sequence or RNA in a plant comprising expressing a recombinant DNA as set forth herein, wherein the at least one heterologous sequence comprises a coding sequence or encodes a functional RNA.

In an embodiment, a method of modulating the expression of a nucleotide sequence of interest in a plant, the method includes expressing a heterologous sequence that is operably linked to a regulatory sequence selected from the group consisting of SEQ ID NOS: 1-6 or a sequence that is at least 95% identical to one of SEQ ID NOS: 1-6. In an embodiment, the heterologous sequence confers an agronomic characteristic selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, nutrient utilization efficiency, nitrogen use efficiency, and salt resistance.

In an embodiment, a method of modulating the expression of a nucleotide sequence of interest in a plant, the method includes expressing a heterologous sequence that is operably linked to a promoter sequence that is at least 90% identical to a sequence selected from the group consisting of SEQ ID NOS: 1-2 in combination with an intron or a 5′UTR functional in a plant cell. In an embodiment, the intron is SEQ ID NO: 3 or 4 or a sequence that is at least 95% identical to SEQ ID NO: 3 or 4. In an embodiment, the 5′UTR is SEQ ID NO: 7 or 8 or a sequence that is at least 95% identical to SEQ ID NO: 7 or 8.

In an embodiment, a plant stably transformed with a recombinant DNA construct includes a regulatory element selected from the group consisting of SEQ ID NOS: 1-6 or a sequence that is at least 90% identical to one of SEQ ID NOS: 1-6 operably linked to a heterologous nucleic acid in the genome of the plant, wherein said regulatory element modulates the expression of the heterologous nucleic acid.

In an embodiment, a plant stably transformed with a recombinant DNA construct that includes a regulatory element selected from the group consisting of SEQ ID NOS: 1-6 or a sequence that is at least 90% identical to one of SEQ ID NOS: 1-6, wherein the plant comprises the regulatory element operably linked to a heterologous nucleic acid in the genome of the plant, wherein the regulatory element modulates the expression of the heterologous nucleic acid. In an embodiment, the plant is a monocot. In an embodiment, the plant is maize. In an embodiment, the heterologous nucleic acid increases yield. In an embodiment, the heterologous nucleic acid increases drought tolerance. In an embodiment, the heterologous nucleic acid encodes a herbicide resistance polypeptide or an insect resistant polypeptide. In an embodiment, the regulatory element comprises a heterologous intron element.

In an embodiment, a plant cell or seed produced from a plant as described herein that includes a regulatory element selected from the group consisting of SEQ ID NOS: 1-6 or a sequence that is at least 90% identical to one of SEQ ID NOS: 1-6, wherein the plant comprises the regulatory element operably linked to a heterologous nucleic acid.

In an embodiment, a method of modifying the expression of an endogenous gene of a plant, the method comprises introducing a regulatory element selected from the group consisting of SEQ ID NOS: 1-6 or a sequence that is at least 90% identical to one of SEQ ID NOS: 1-6 such that the introduced regulatory element is operably linked to modify the expression of the endogenous gene. In an embodiment, the regulatory element is introduced through genome editing. In an embodiment, the genome editing is performed through guided Cas9 endonuclease. In an embodiment, a plant is produced by this method. In an embodiment, the regulatory element is a fragment of one of nucleic acid sequences represented by SEQ ID NOS: 1-6 and comprises at least about 100 contiguous nucleic acids of one of SEQ ID NOS: 1-6.

A recombinant DNA construct includes at least one heterologous nucleotide sequence operably linked to a promoter wherein said promoter comprises the nucleotide sequence set forth in SEQ ID NOS: 1-2 and 5-6, or said promoter comprises a functional fragment of the nucleotide sequence set forth in SEQ ID NOS: 1-2 and 5-6, or wherein said promoter comprises a nucleotide sequence having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity, based on the Clustal V method of alignment with pairwise alignment default parameters (KTUPLE=2, GAP PENALTY=5, WINDOW=4 and DIAGONALS SAVED=4), when compared to the nucleotide sequence of SEQ ID NOS: 1-2 and 5-6.

In another embodiment, this disclosure concerns a recombinant DNA construct comprising a nucleotide sequence comprising any of the sequences set forth in SEQ ID NOS: 1-8, or a functional fragment thereof, operably linked to at least one heterologous sequence, wherein said nucleotide sequence is a constitutive promoter.

In another embodiment, this disclosure concerns a recombinant DNA construct comprising a nucleotide sequence having at least 95% identity, based on the Clustal V method of alignment with pairwise alignment default parameters (KTUPLE=2, GAP PENALTY=5, WINDOW=4 and DIAGONALS SAVED=4), when compared to the sequence set forth in SEQ ID NOS: 1-2 and 5-6.

In another embodiment, this disclosure concerns a recombinant DNA construct comprising at least one heterologous nucleotide sequence operably linked to a Sorghum or Setaria GOS2 regulatory element as set forth in SEQ ID NOS:1, 2, 5, or 6, wherein said promoter comprises a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 11311, 1132, 1133, 1134, 1135, 1136, 1137, 1138, 1139, 1140, 1141, 1142, 1143, 1144, 1145, 1146, 1147, 1148, 1149, 1150, 11511, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, or 1246 consecutive nucleotides or up to the region containing a TATA box or an equivalent motif thereof, of SEQ ID NOS:1, 2, 5, or 6. This disclosure also concerns a recombinant DNA construct of the embodiments disclosed herein, wherein the promoter is a constitutive promoter.

In another embodiment, this disclosure concerns a cell, plant, or seed comprising a recombinant DNA construct of the present disclosure.

In another embodiment, this disclosure concerns plants comprising this recombinant DNA construct and seeds obtained from such plants.

In another embodiment, this disclosure concerns a method of altering (increasing or decreasing) expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing the transformed plant cell wherein the expression of the heterologous nucleic acid fragment is increased or decreased.

In another embodiment, this disclosure concerns a recombinant DNA construct comprising a Sorghum or Setaria GOS2 regulatory element.

In another embodiment, this disclosure concerns a method of altering a marketable plant trait. The marketable plant trait concerns genes and proteins involved in disease resistance, herbicide resistance, insect resistance, carbohydrate metabolism, fatty acid metabolism, amino acid metabolism, plant development, plant growth regulation, yield improvement, drought resistance, cold resistance, heat resistance, and salt resistance.

In another embodiment, this disclosure concerns a recombinant DNA construct comprising a heterologous nucleotide sequence. The heterologous nucleotide sequence encodes a protein involved in disease resistance, herbicide resistance, insect resistance; carbohydrate metabolism, fatty acid metabolism, amino acid metabolism, plant development, plant growth regulation, yield improvement, drought resistance, cold resistance, heat resistance, or salt resistance in plants.

### BRIEF DESCRIPTION OF THE DRAWINGS AND SEQUENCE LISTINGS

The disclosure can be more fully understood from the following detailed description and the accompanying drawings and Sequence Listing that form a part of this application.

FIG. 2A-2B-2C show an alignment of GOS2 regulatory sequences from maize (includes SEQ ID NOS: 9, 10 and 11), Sorghum (includes SEQ ID NOs: 1, 3 and 7) and Setaria (includes SEQ ID NOs: 2, 4 and 8). The TATA box is shown in a rectangular box; the intron splice sites are underlined; the intron sequences are in italics; and the ATG start codon is shown as grey highlights.

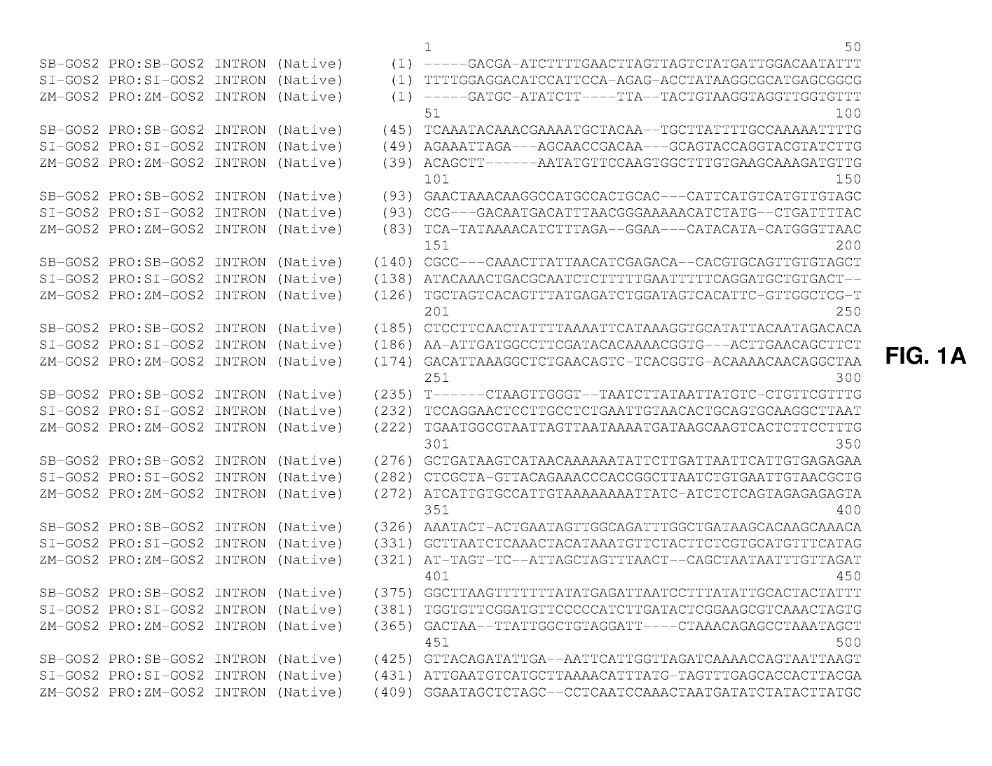

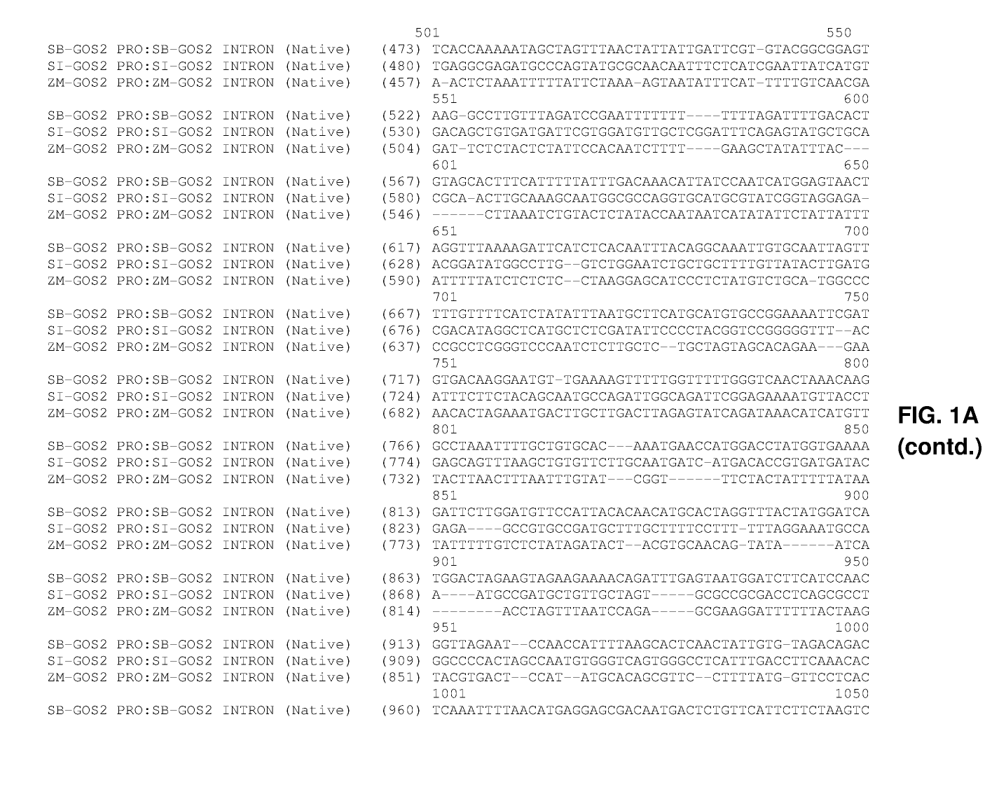

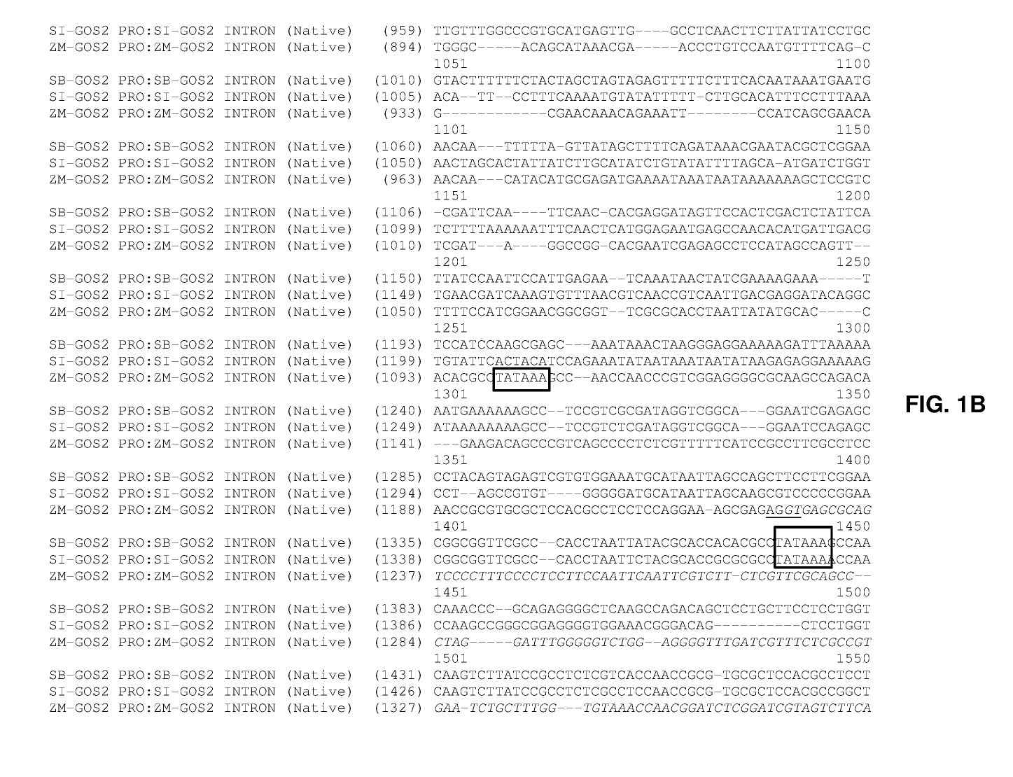

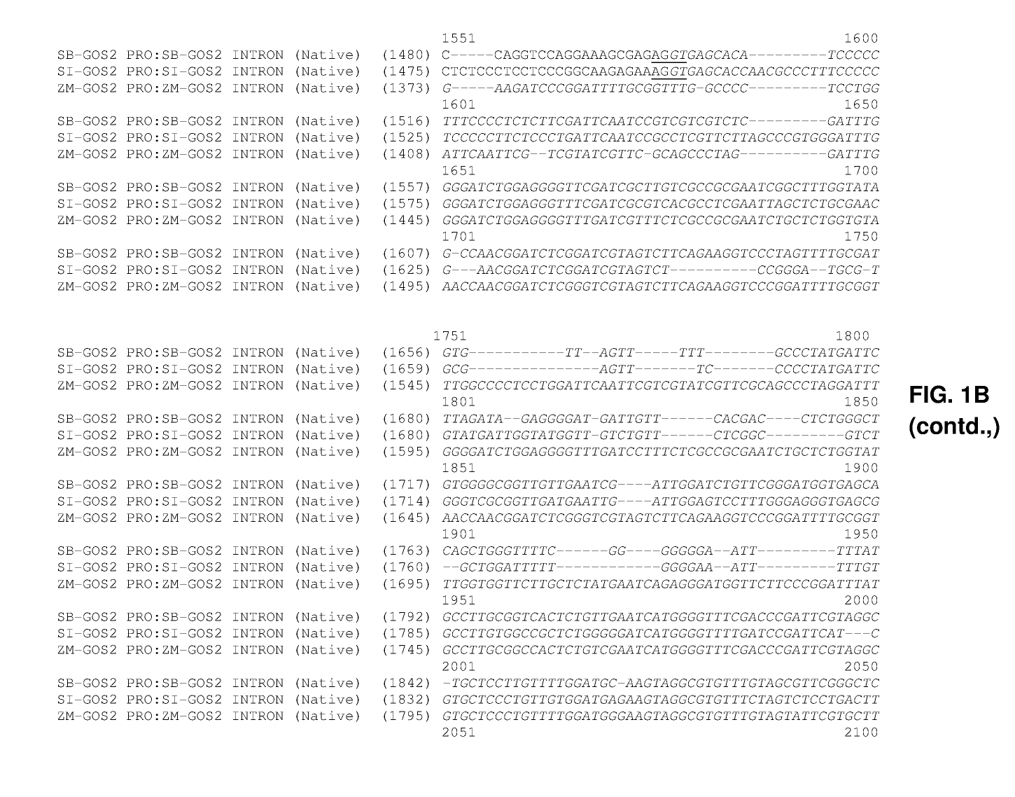

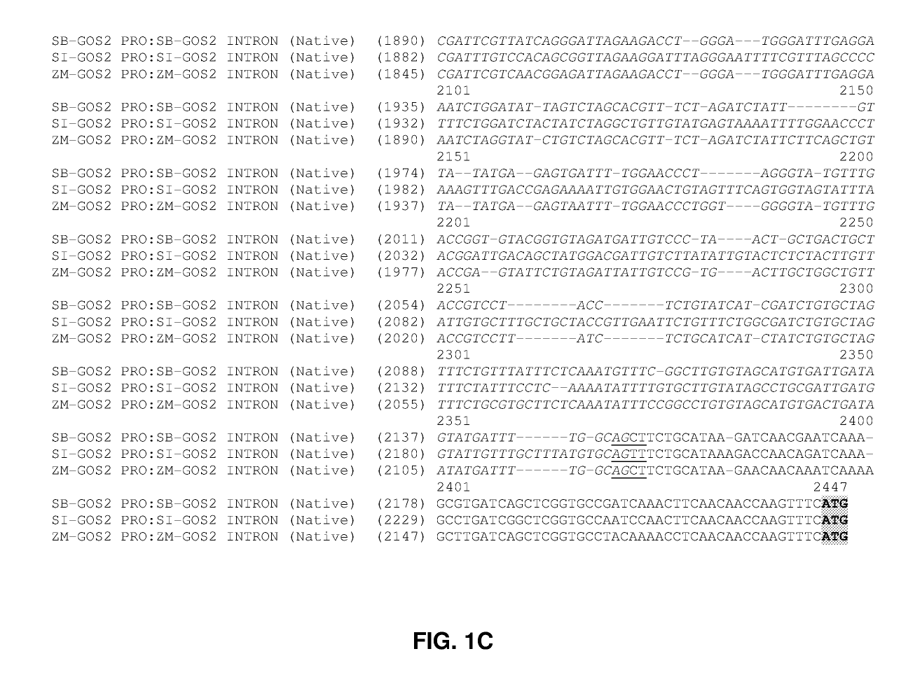

The sequence descriptions summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| SEQ ID NO: | Description |
|---|---|
| 1 | Sorghum GOS2 promoter |
| 2 | Setaria GOS2 promoter |
| 3 | Sorghum GOS2 intron 1 |
| 4 | Setaria GOS2 intron 1 |
| 5 | Sorghum GOS2 promoter + intron 1 + 5′UTR |
| 6 | Setaria GOS2 promoter + intron 1 + 5′UTR |
| 7 | Sorghum GOS2 5′UTR |
| 8 | Setaria GOS2 5′UTR |
| 9 | Maize GOS2 promoter |
| 10 | Maize GOS2 intron |
| 11 | Maize GOS2 5′ UTR |

### DETAILED DESCRIPTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

In the context of this disclosure, a number of terms shall be utilized.

An “isolated polynucleotide” generally refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Sorghum GOS2 promoter”, refer to the promoter of a Sorghum bicolor GOS2 gene with similarity to the rice GOS2 gene (de Pater et al., (1992) Plant J. November; 2(6):837-44)). The term “Sorghum GOS2 promoter” encompasses a sequence upstream of the translation initiation site, excluding an intron 1 sequence. Minor sequence alterations for restriction sites were included to facilitate cloning.

“Setaria GOS2 promoter”, refer to the promoter of a Setaria italica GOS2 gene with similarity to the rice GOS2 gene (de Pater et al., (1992) Plant J. November; 2(6):837-44)). The term “Setaria GOS2 promoter” encompasses a sequence upstream of the translation initiation site, excluding an intron 1 sequence. Minor sequence alterations for restriction sites were included to facilitate cloning.

A regulatory element generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

An enhancer element is any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” generally refers to a promoter whose activity is determined by developmental events.

“Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

The promoter nucleotide sequences and methods disclosed herein are useful in regulating constitutive expression of any heterologous nucleotide sequences in a host plant in order to alter the phenotype of a plant.

A “heterologous nucleotide sequence” generally refers to a sequence that is not naturally occurring with the plant promoter sequence of the disclosure. While this nucleotide sequence is heterologous to the promoter sequence, it may be homologous, or native, or heterologous, or foreign, to the plant host. However, it is recognized that the instant promoters may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

The present disclosure encompasses recombinant DNA constructs comprising functional fragments of the promoter sequences disclosed herein.

A “functional fragment” refer to a portion or subsequence of the promoter sequence of the present disclosure in which the ability to initiate transcription or drive gene expression (such as to produce a certain phenotype) is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

A nucleic acid fragment that is functionally equivalent to the promoter of the present disclosure is any nucleic acid fragment that is capable of controlling the expression of a coding sequence or functional RNA in a similar manner to the promoter of the present disclosure.

In an embodiment of the present disclosure, the promoters disclosed herein can be modified. Those skilled in the art can create promoters that have variations in the polynucleotide sequence. The polynucleotide sequence of the promoters of the present disclosure as shown in SEQ ID NOS: 1-2, may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

A “variant promoter”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Methods for construction of chimeric and variant promoters of the present disclosure include, but are not limited to, combining control elements of different promoters or duplicating portions or regions of a promoter. Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In some aspects of the present disclosure, the promoter fragments can comprise at least about 20 contiguous nucleotides, or at least about 50 contiguous nucleotides, or at least about 75 contiguous nucleotides, or at least about 100 contiguous nucleotides, or at least about 150 contiguous nucleotides, or at least about 200 contiguous nucleotides of SEQ ID NOS: 1-2 and 5-6. In another aspect of the present disclosure, the promoter fragments can comprise at least about 250 contiguous nucleotides, or at least about 300 contiguous nucleotides, or at least about 350 contiguous nucleotides, or at least about 400 contiguous nucleotides, or at least about 450 contiguous nucleotides, or at least about 500 contiguous nucleotides, or at least about 550 contiguous nucleotides, or at least about 600 contiguous nucleotides, or at least about 650 contiguous nucleotides, or at least about 700 contiguous nucleotides, or at least about 750 contiguous nucleotides, or at least about 800 contiguous nucleotides, or at least about 850 contiguous nucleotides, or at least about 900 contiguous nucleotides, or at least about 950 contiguous nucleotides, or at least about 1000 contiguous nucleotides, or at least about 1050 contiguous nucleotides, of SEQ ID NO:1. In another aspect, a promoter fragment is the nucleotide sequence set forth in SEQ ID NO:2, SEQ ID NO:5, or SEQ ID NO:6. The nucleotides of such fragments generally comprise the TATA recognition sequence of the particular promoter sequence. Such fragments may be obtained by use of restriction enzymes to cleave the naturally occurring promoter nucleotide sequences disclosed herein, by synthesizing a nucleotide sequence from the naturally occurring promoter DNA sequence, or may be obtained through the use of PCR technology.

The terms “full complement” and “full-length complement” are used interchangeably herein, and refer to a complement of a given nucleotide sequence, wherein the complement and the nucleotide sequence consist of the same number of nucleotides and are 100% complementary.

The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

The transitional phrase “consisting essentially of” generally refers to a composition, method that includes materials, steps, features, components, or elements, in addition to those literally disclosed, provided that these additional materials, steps, features, components, or elements do not materially affect the basic and novel characteristic(s) of the claimed subject matter.

The isolated promoter sequence comprised in the recombinant DNA construct of the present disclosure can be modified to provide a range of constitutive expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present disclosure can provide for a range of constitutive expression of the heterologous nucleotide sequence. Thus, they may be modified to be weak constitutive promoters or strong constitutive promoters. Generally, by “weak promoter” is intended a promoter that drives expression of a coding sequence at a low level. By “low level” is intended levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts. Similarly, a “moderate constitutive” promoter is somewhat weaker than a strong constitutive promoter like the maize ubiquitin promoter.

Moreover, the skilled artisan recognizes that substantially similar nucleic acid sequences encompassed by this disclosure are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the disclosure. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U. K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 71% to 100%, such as 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In one embodiment, the isolated promoter sequence comprised in the recombinant DNA construct of the present disclosure comprises a nucleotide sequence having at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity, based on the Clustal V method of alignment with pairwise alignment default parameters (KTUPLE=2, GAP PENALTY=5, WINDOW=4 and DIAGONALS SAVED=4), when compared to the nucleotide sequence of SEQ ID NOS: 1-2 and 5-6. It is known to one of skilled in the art that a 5′ UTR region can be altered (deletion or substitutions of bases) or replaced by an alternative 5′UTR while maintaining promoter activity.

A “substantially similar sequence” generally refers to variants of the disclosed sequences such as those that result from site-directed mutagenesis, as well as synthetically derived sequences. A substantially similar sequence of the present disclosure also generally refers to those fragments of a particular promoter nucleotide sequence disclosed herein that operate to promote the constitutive expression of an operably linked heterologous nucleic acid fragment. These promoter fragments comprise at least about 20 contiguous nucleotides, at least about 50 contiguous nucleotides, at least about 75 contiguous nucleotides, preferably at least about 100 contiguous nucleotides of the particular promoter nucleotide sequence disclosed herein or a sequence that is at least 95 to about 99% identical to such contiguous sequences. The nucleotides of such fragments will usually include the TATA recognition sequence (or CAAT box or a CCAAT) of the particular promoter sequence. Such fragments may be obtained by use of restriction enzymes to cleave the naturally occurring promoter nucleotide sequences disclosed herein;

by synthesizing a nucleotide sequence from the naturally occurring promoter DNA sequence; or may be obtained through the use of PCR technology. Variants of these promoter fragments, such as those resulting from site-directed mutagenesis, are encompassed by the compositions of the present disclosure.

“Codon degeneracy” generally refers to divergence in the genetic code permitting variation of the nucleotide sequence without affecting the amino acid sequence of an encoded polypeptide. Accordingly, the instant disclosure relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In one embodiment the % sequence identity is determined over the entire length of the molecule (nucleotide or amino acid).

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN generally refers to a BLAST program that compares a nucleotide query sequence against a nucleotide sequence database.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” generally refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

“Chimeric gene” or “recombinant expression construct”, which are used interchangeably, includes any gene that is not a native gene, comprising regulatory and coding sequences that are not found together in nature. Accordingly, a chimeric gene may comprise regulatory sequences and coding sequences that are derived from different sources.

“Coding sequence” generally refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” generally refers to a product resulting from RNA polymerase-catalyzed transcription of a DNA sequence. When an RNA transcript is a perfect complimentary copy of a DNA sequence, it is referred to as a primary transcript or it may be a RNA sequence derived from posttranscriptional processing of a primary transcript and is referred to as a mature RNA. “Messenger RNA” (“mRNA”) generally refers to RNA that is without introns and that can be translated into protein by the cell. “cDNA” generally refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA generally refers to RNA transcript that includes mRNA and so can be translated into protein within a cell or in vitro. “Antisense RNA” generally refers to a RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” generally refers to antisense RNA, ribozyme RNA, or other RNA that may not be translated but yet has an effect on cellular processes.

The term “operably linked” or “functionally linked” generally refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, generally refers to the production of a functional end-product e.g., an mRNA or a protein (precursor or mature).

The term “expression cassette” as used herein, generally refers to a discrete nucleic acid fragment into which a nucleic acid sequence or fragment can be cloned or synthesized through molecular biology techniques.

Expression or overexpression of a gene involves transcription of the gene and translation of the mRNA into a precursor or mature protein. “Antisense inhibition” generally refers to the production of antisense RNA transcripts capable of suppressing the expression of the target protein. “Overexpression” generally refers to the production of a gene product in transgenic organisms that exceeds levels of production in normal or non-transformed organisms. “Co-suppression” generally refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at post-transcriptional level.

As stated herein, “suppression” includes a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” generally refers to an enzyme that is produced naturally in a non-transgenic or wild type cell. The terms “non-transgenic” and “wild type” are used interchangeably herein.

“Altering expression” or “modulating expression” generally refers to the production of gene product(s) in plants in amounts or proportions that differ significantly from the amount of the gene product(s) produced by the corresponding wild-type plants (i.e., expression is increased or decreased).

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

Transgenic includes any cell, cell line, callus, tissue, plant part or plant, the genome of which has been altered by the presence of a heterologous nucleic acid, such as a recombinant DNA construct.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

“Progeny” comprises any subsequent generation of a plant.

A transgenic plant includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by genome editing procedures that do not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

“Transient expression” generally refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Various changes in phenotype are of interest including, but not limited to, modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic characteristics and traits such as yield and heterosis increase, the choice of genes for transformation will change accordingly. General categories of genes of interest include, but are not limited to, those genes involved in information, such as zinc fingers, those involved in communication, such as kinases, and those involved in housekeeping, such as heat shock proteins. More specific categories, for example, include, but are not limited to, genes encoding important traits for agronomics, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

Disease and/or insect resistance genes may encode resistance to pests that have great yield drag such as for example, Northern Corn Leaf Blight, head smut, anthracnose, soybean mosaic virus, soybean cyst nematode, root-knot nematode, brown leaf spot, Downy mildew, purple seed stain, seed decay and seedling diseases caused commonly by the fungi—Pythium sp., Phytophthora sp., Rhizoctonia sp., Diaporthe sp. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorsulfuron. Glyphosate acetyl transferase (GAT) is an N-acetyltransferase from Bacillus licheniformis that was optimized by gene shuffling for acetylation of the broad spectrum herbicide, glyphosate, forming the basis of a novel mechanism of glyphosate tolerance in transgenic plants (Castle et al. (2004) Science 304, 1151-1154).

Genes involved in plant growth and development have been identified in plants. One such gene, which is involved in cytokinin biosynthesis, is isopentenyl transferase (IPT). Cytokinin plays a critical role in plant growth and development by stimulating cell division and cell differentiation (Sun et al. (2003), Plant Physiol. 131: 167-176).

In certain embodiments, the present disclosure contemplates the transformation of a recipient cell with more than one advantageous gene. Two or more genes can be supplied in a single transformation event using either distinct gene-encoding vectors, or a single vector incorporating two or more gene coding sequences. Any two or more genes of any description, such as those conferring herbicide, insect, disease (viral, bacterial, fungal, and nematode), or drought resistance, oil quantity and quality, or those increasing yield or nutritional quality may be employed as desired.

This disclosure concerns a recombinant DNA construct comprising an isolated nucleic acid fragment comprising a constitutive Sorghum or Setaria GOS2 promoter. This disclosure also concerns a recombinant DNA construct comprising a promoter wherein said promoter consists essentially of the nucleotide sequence set forth in SEQ ID NO:1 or 2, or an isolated polynucleotide comprising a promoter wherein said promoter comprises the nucleotide sequence set forth in SEQ ID NOS: 1-2 and 5-6 or a functional fragment of SEQ ID NOS: 1-2 and 5-6.

It is clear from the disclosure set forth herein that one of ordinary skill in the art could perform the following procedure:

1) operably linking the nucleic acid fragments containing the Sorghum or Setaria GOS2 promoter, intron or the 5′UTR sequences to a suitable reporter gene; there are a variety of reporter genes that are well known to those skilled in the art, including the bacterial GUS gene, the firefly luciferase gene, and the cyan, green, red, and yellow fluorescent protein genes; any gene for which an easy and reliable assay is available can serve as the reporter gene.

2) transforming Sorghum or Setaria GOS2 promoter, intron or the 5′UTR sequences:reporter gene expression cassettes into an appropriate plant for expression of the promoter. There are a variety of appropriate plants which can be used as a host for transformation that are well known to those skilled in the art, including the dicots, Arabidopsis, tobacco, soybean, oilseed rape, peanut, sunflower, safflower, cotton, tomato, potato, cocoa and the monocots, corn, wheat, rice, barley and palm.

3) testing for expression of the Sorghum or Setaria GOS2 promoter, intron or the 5′UTR sequences in various cell types of transgenic plant tissues, e.g., leaves, roots, flowers, seeds, transformed with the chimeric Sorghum or Setaria GOS2 promoter, intron or the 5′UTR sequences:reporter gene expression cassette by assaying for expression of the reporter gene product.

In another aspect, this disclosure concerns a recombinant DNA construct comprising at least one heterologous nucleic acid fragment operably linked to any promoter, or combination of promoter elements, of the present disclosure. Recombinant DNA constructs can be constructed by operably linking the nucleic acid fragment of the disclosure Sorghum or Setaria GOS2 promoter or a fragment that is substantially similar and functionally equivalent to any portion of the nucleotide sequence set forth in SEQ ID NOS: 1-2 and 5-6 to a heterologous nucleic acid fragment. Any heterologous nucleic acid fragment can be used to practice the disclosure. The selection will depend upon the desired application or phenotype to be achieved. The various nucleic acid sequences can be manipulated so as to provide for the nucleic acid sequences in the proper orientation. It is believed that various combinations of promoter elements as described herein may be useful in practicing the present disclosure.

In another aspect, this disclosure concerns a recombinant DNA construct comprising at least one gene that provides drought tolerance operably linked to Sorghum or Setaria GOS2 promoter or a fragment, or combination of promoter elements, of the present disclosure. In another aspect, this disclosure concerns a recombinant DNA construct comprising at least one gene that provides insect resistance operably linked to Sorghum or Setaria GOS2 promoter or a fragment, or combination of promoter elements, of the present disclosure. In another aspect, this disclosure concerns a recombinant DNA construct comprising at least one gene that increases nitrogen use efficiency and/or yield, operably linked to Sorghum or Setaria GOS2 promoter or a fragment, or combination of promoter elements, of the present disclosure. In another aspect, this disclosure concerns a recombinant DNA construct comprising at least one gene that provides herbicide resistance operably linked to Sorghum or Setaria GOS2 promoter or a fragment, or combination of promoter elements, of the present disclosure.

In another embodiment, this disclosure concerns host cells comprising either the recombinant DNA constructs of the disclosure as described herein or isolated polynucleotides of the disclosure as described herein. Examples of host cells which can be used to practice the disclosure include, but are not limited to, yeast, bacteria, and plants.

Plasmid vectors comprising the instant recombinant DNA construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

In general, methods to modify or alter the host endogenous genomic DNA are available. This includes altering the host native DNA sequence or a pre-existing recombinant sequence including regulatory elements, coding and non-coding sequences. These methods are also useful in targeting nucleic acids to pre-engineered target recognition sequences in the genome. In an embodiment, one or more of the regulatory elements associated with the GOS2 promoters of Sorgum and Setaria disclosed herein are inserted at a desired location in the maize genome. For example, such insertion may occur to drive the expression of an endogenous maize gene involved in drought, cold, frost, nitrogen use efficiency or yield increase. In an embodiment, the endogenous gene is a transcription factor involved in modulating one or more agronomic characteristics of the host plant such as a maize plant. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1).

In an embodiment, through genome editing approaches described herein and those available to one of ordinary skill in the art, specific motifs of one or more regulatory elements of the Sorghum and Setaria GOS2 promoters disclosed herein can be engineered to modulate the expression of one or more host plant endogenous genes.

Methods for transforming dicots, primarily by use of Agrobacterium tumefaciens, and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C.A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

The level of activity of the Sorghum or Setaria GOS2 promoter is weaker than that of many known strong promoters, such as the CaMV 35S promoter (Atanassova et al., Plant Mol. Biol. 37:275-285 (1998); Battraw and Hall, Plant Mol. Biol. 15:527-538 (1990); Holtorf et al., Plant Mol. Biol. 29:637-646 (1995); Jefferson et al., EMBO J. 6:3901-3907 (1987); Wilmink et al., Plant Mol. Biol. 28:949-955 (1995)). Universal moderate expression of chimeric genes in most plant cells makes the Sorghum or Setaria GOS2 promoter of the instant disclosure especially useful when moderate constitutive expression of a target heterologous nucleic acid fragment is required.

Another general application of the Sorghum or Setaria GOS2 promoter of the disclosure is to construct chimeric genes that can be used to reduce expression of at least one heterologous nucleic acid fragment in a plant cell. To accomplish this, a chimeric gene designed for gene silencing of a heterologous nucleic acid fragment can be constructed by linking the fragment to the Sorghum or Setaria GOS2 promoter of the present disclosure. Alternatively, a chimeric gene designed to express antisense RNA for a heterologous nucleic acid fragment can be constructed by linking the fragment in reverse orientation to the Sorghum or Setaria GOS2 promoter of the present disclosure. Either the cosuppression or antisense chimeric gene can be introduced into plants via transformation. Transformants wherein expression of the heterologous nucleic acid fragment is decreased or eliminated are then selected.

This disclosure also concerns a method of altering (increasing or decreasing) the expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing a transformed plant cell wherein the expression of the heterologous nucleic acid fragment is increased or decreased.

Transformation and selection can be accomplished using methods well-known to those skilled in the art including, but not limited to, the methods described herein.

### EXAMPLES

The present disclosure is further defined in the following Examples, in which parts and percentages are by weight and degrees are Celsius, unless otherwise stated. Sequences of promoters, cDNA, adaptors, and primers listed in this disclosure all are in the 5′ to 3′ orientation unless described otherwise. Techniques in molecular biology were typically performed as described in Ausubel, F. M. et al., In Current Protocols in Molecular Biology; John Wiley and Sons: New York, 1990 or Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”). It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Expression Analysis of Sorghum and Setaria GOS2 Promoter Sequences

Promoter sequences from Sorghum and Setaria were identified and appropriate transformation vectors with a reporter gene (e.g., GUS) were constructed. Sequence alignments are shown in FIGS. 1A, 1B, and 1C.

TABLE 2
Promoter element sequences and accompanying introns

| Promoter Element | Intron |
|---|---|
| SB-GOS2 PRO (MOD1) | SB-GOS2 INTRON1 |
| (SEQ ID NO: 1) | (SEQ ID NO: 3) |
| SI-GOS2 PRO (MOD1) | SI-GOS2 INTRON1 (MOD1) |
| (SEQ ID NO: 2) | (SEQ ID NO: 4) |

Expression cassettes containing Sorghum GOS2 promoter sequences along with Sorghum GOS2 intron were constructed using standard molecular biology techniques. The Sorghum GOS2 promoter and intron sequences are disclosed herein (see Table 1 and the accompanying sequence listing file). Similarly, expression cassettes containing Setaria GOS2 promoter sequences along with Sorghum GOS2 intron were constructed using standard molecular biology techniques. In both sets of expression cassettes, a standard GUS gene was used as a reporter gene to measure the promoter activity.

Maize transformation with plant transformation vectors containing the expression cassettes having the Sorghum and Setaria GOS2 promoters as described herein was conducted. Eight events from each construct were evaluated for expression of the GUS gene following standard GUS staining procedures. Leaf samples from T0 maize plants expressing the GUS (under the tested promoter/intron combination) were collected and analyzed for GUS gene expression. The results are shown in Table 3.

TABLE 3
Expression Analysis of the Sorghum and Setaria Promoters

| | No. of leaf | |
|---|---|---|
| | samples | |
| | showing | |
| Promoter - Intron | positive GUS | Remarks (qualitative |
| Combination | staining | strength of staining) |
| ZM-GOS2 PRO:ZM-GOS2 | 8/8 | +++ |
| INTRON1 | | |
| SB-GOS2 PRO:SB-GOS2 | 6/8 | +++ |
| INTRON1 | | |
| SI-GOS2 PRO:SI-GOS2 | 6/8 | ++++ |
| INTRON1 | | |
| UBI1ZM PRO:UBI1ZM | 7/8 | ++++ |
| INTRON1 | | |

Sorghum GOS2 promoter in combination with the Sorghum GOS2 intron driving GUS gene expression in maize T0 plants was analyzed along with comparators such as for example, the maize GOS2 promoter (with maize GOS2 intron) and the maize ubiquitin promoter (with maize ubiquitin intron). Similarly, Setaria GOS2 promoter in combination with the Setaria GOS2 intron driving GUS gene expression in maize T0 plants was analyzed along with the same comparators. Results are shown in Table 3 and Table 4. Positive GUS staining was observed for the constructs tested with the GOS2 regulatory elements described herein in this Example 1.

TABLE 4
Expression Analysis of the Sorghum and Setaria Promoters (quantitative
GUS staining results)

| | | Leaf GUS Score | Root GUS Score | |
|---|---|---|---|---|
| Plasmid/ | | (Avg) (nmoles | (Avg) (nmoles | No. of |
| Construct | Promoter | MU/mg TP/hr) | MU/mg TP/hr) | events |
| Empty | None | 0 | 0 | N/A (2 |
| Control (No | | | | samples) |
| Tissue) | | | | |
| Neg_Ctl | None | 0 | 0 | N/A (2 |
| (Null) | | | | samples) |
| PHP-016 | ZM-GOS2 PRO:ZM- | 110 | 233 | 7 |
| | GOS2 INTRON1 | | | |
| PHP-021 | SB-GOS2 PRO:SB- | 151 | 298 | 13 |
| | GOS2 INTRON1 | | | |
| PHP-022 | SI-GOS2 PRO:SI- | 198 | 412 | 4 |
| | GOS2 INTRON1 | | | |
| PHP-033 | UBI1ZM PRO: | 696 | 1634 | 14 |
| | UBI1ZM INTRON | | | |

For the Sorghum GOS2 promoter in combination with the Sorghum GOS2 intron, 6 out of 8 events showed positive GUS staining in the maize leaves. The intensity of the GUS stain was similar to those observed for the maize GOS2 promoter (with maize GOS2 intron) in the maize leaves. For the Setaria GOS2 promoter in combination with the Setaria GOS2 intron, 6 out of 8 events showed positive GUS staining in the maize leaves. The GUS staining was more intense than that of the maize GOS2 promoter and was similar to those observed for the maize ubiquitin promoter (with maize ubiquitin intron) in the maize leaves.

Control maize leaves not containing the tested regulatory elements (e.g., GOS2, and ubiquitin) did not show GUS staining.

As demonstrated in Table 4, average leaf and root GUS scores for T1 single copy events in corn expressing the regulatory elements show that the Sorghum and Setaria GOS2 promoters tested have constitutive expression pattern. One sample per event was tested for each leaf and root expression analysis. For GUS staining and scoring, see Gallagher, S.R. (1992) Quantitation of GUS activity by fluorometry. In GUS Protocols: using the GUS gene as a reporter of gene expression (Gallagher, S.R., ed): Academic Press, Inc., New York, pp. 47-59.

These results demonstrate that the regulatory elements derived from Sorghum and Setaria possess promoter activity as evidenced by the stable expression of the heterologous GUS gene in maize leaves.

### Example 2

### Sorghum and Setaria GOS2 Regulatory Element Fragments Showing Promoter Activity

To define the transcriptional elements controlling the GOS2 promoter activity, the full length regulatory element of Sorghum GOS2 (SEQ ID NO: 6) and Setaria GOS2 (SEQ ID NO: 6) are further truncated or deleted at the 3′ or the 5′ end to generate deletion fragments of varying sizes, e.g, 300 bp, 400 bp, 500 bp, 600 bp up to about 1000 bp. PCR amplification is performed based on the full length sequences provided herein.

One of the standard methods for identifying motifs controlling the characteristics of a promoter's expression capabilities involves creating a truncation or deletion series of sequences driving a reporter gene marker such as GUS, GFP, luciferase, or any other suitable fluorescent protein. One typical approach begins with a deletion series removing ˜10 percent of the promoter sequence beginning at the 5′ end. Expression of the marker gene for each truncation is quantified and observations made regarding changes in expression levels. Once a distinction has been made between truncations that have no effect and those that do, a deletion series can be made to further tease out which precise sequences have an impact on expression levels.

For example, if a 2 kb promoter sequence is the initial starting sequence, sequences with lengths of 2 kb-1 kb show some the same level of expression, it is generally expected that no significant motifs affecting expression in the tissue tested are present in the most 5′ 1 kb of sequence. If it is found that truncations under 0.4 kb lose all function, it is determined that the minimal promoter for expression is about 0.4 kb. Then, a deletion series is created where a 100 bp region is sequentially removed, from within the remaining 1 kb, in a stepwise fashion until a deletion series is created where each region of the remaining 1 KB sequence, upstream of the “minimal promoter” has been removed for testing. Thus, for example, 5 new deletion series all with a length of about 0.9 kb (0.4 kb of minimal promoter+0.5 kb of upstream region) are constructed for testing in the same fashion as before.

These deletion series can be tested through stable transformation of a suitable plant or through transient expression analysis e.g., using leaf disks.

### Example 3

### Endogenous Gene Expression Modification Through Genome Editing

In an embodiment, the regulatory elements set forth in SEQ ID NOS: 1-8 or fragments thereof, and compositions comprising said sequences, can be inserted in operable linkage with an endogenous gene by genome editing using a double-stranded break inducing agent, such as a guided Cas9 endonuclease. Based on the availability of the genetic loci sequence information guide RNAs are designed to target a particular endogenous gene. For example maize genes involved in improving agronomic characteristics of a maize plant are suitable targets.

Guided Cas9 endonucleases are derived from CRISPR loci (Clustered Regularly Interspaced Short Palindromic Repeats) (also known as SPIDRs—SPacer Interspersed Direct Repeats) which are a family of recently described DNA loci. CRISPR loci are characterized by short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas endonuclease relates to a Cas protein encoded by a Cas gene, wherein the Cas protein is capable of introducing a double strand break into a DNA target sequence. The Cas endonuclease is guided by a guide polynucleotide to recognize and optionally introduce a double strand break at a specific target site into the genome of a cell (U.S. Application Publication No. 2015/0082478). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

In one embodiment, the methods comprise modifying the expression of an endogenous gene in a cell by introducing the regulatory elements herein in operable linkage with an endogenous gene. The regulatory elements can be introduced in operable linkage to an endogenous gene using any genome editing technique, including, but not limited to use of a double-stranded break inducing agent, such as guided Cas9/CRISPR system, Zinc finger nucleases, TALENs. See Ma et al (2014), Scientific Reports, 4: 4489; Daimon et al (2013), Development, Growth, and Differentiation, 56(1): 14-25; and Eggleston et al (2001) BMC Genetics, 2:11.

1. A recombinant DNA construct comprising a polynucleotide sequence comprising any of the sequences set forth in SEQ ID NOS: 1-8, or a fragment that comprises at least about 50-100 contiguous nucleotides of SEQ ID NO: 1-6, operably linked to at least one heterologous nucleic acid sequence.

2. The recombinant DNA construct of claim 1, wherein the polynucleotide sequence has at least 95% identity, based on the Clustal V method of alignment with pairwise alignment default parameters (KTUPLE=2, GAP PENALTY=5, WINDOW=4 and DIAGONALS SAVED=4), when compared to any of the sequences set forth in SEQ ID NOS: 1-8.

3. A vector comprising the recombinant DNA construct of claim 1.

4. A cell comprising the recombinant DNA construct of claim 1.

5. The cell of claim 4, wherein the cell is a plant cell.

6. A plant having stably incorporated into its genome the recombinant DNA construct of claim 1.

7. The plant of claim 6 wherein said plant is a monocot plant.

8. The plant of claim 7 wherein the plant is maize.

9. A seed produced by the plant of claim 7, wherein the seed comprises the recombinant DNA construct.

10. The recombinant DNA construct of claim 1 wherein the at least one heterologous nucleic acid sequence comprises a genetic sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

11. (canceled)

12. (canceled)

13. A method of modulating the expression of a nucleotide sequence of interest in a plant, the method comprising expressing a heterologous sequence that is operably linked to a regulatory sequence selected from the group consisting of SEQ ID NOS: 1-6 or a sequence that is at least 95% identical to one of SEQ ID NOS: 1-6.

14. The method of claim 13, wherein the heterologous sequence confers an agronomic characteristic selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, nutrient utilization efficiency, nitrogen use efficiency, and salt resistance.

15. The method of claim 13, wherein regulatory sequence comprises an intron or a 5′UTR functional in a plant cell.

16. The method of claim 15 wherein the intron is SEQ ID NO: 3 or 4 or a sequence that is at least 95% identical to SEQ ID NO: 3 or 4.

17. The method of claim 15 wherein the 5′UTR is SEQ ID NO: 7 or 8 or a sequence that is at least 95% identical to SEQ ID NO: 7 or 8.

18. (canceled)

19. A method of modifying the expression of an endogenous gene of a plant, the method comprising introducing a regulatory element selected from the group consisting of SEQ ID NOS: 1-6 or a sequence that is at least 90% identical to one of SEQ ID NOS: 1-6 such that the introduced regulatory element is operably linked to modify the expression of the endogenous gene.

20. The method of claim 19, wherein the regulatory element is introduced through genome editing.

21. The method of claim 20, wherein the genome editing is performed through a RNA guided endonuclease.

22. A plant produced by the method of claim 19.

23. (canceled)

24. The plant of claim 18 is a monocot.

25. (canceled)

26. (canceled)

27. (canceled)

28. (canceled)

29. (canceled)

30. (canceled)
